SEQUENCE LISTING (54 sequences)

SEQ ID NO: 1 - DNA, 36 bp, synthetic construct
  source 1..36: mol_type = other DNA
aaaaggtggtggggggggttggtagggtgtcttcta

SEQ ID NO: 2 - DNA, 28 bp, synthetic construct
  source 1..28: mol_type = other DNA
acaggggtgtggggacaggggtgtgggg

SEQ ID NO: 3 - DNA, 15 bp, synthetic construct
  source 1..15: mol_type = other DNA
ggttggtgtggttgg

SEQ ID NO: 4 - DNA, 29 bp, synthetic construct
  source 1..29: mol_type = other DNA
agtccgtggtagggcaggttggggtgact

SEQ ID NO: 5 - DNA, 14 bp, synthetic construct
  source 1..14: mol_type = other DNA
aggttggggtgact

SEQ ID NO: 6 - DNA, 15 bp, synthetic construct
  source 1..15: mol_type = other DNA
agtccgtggtagggc

SEQ ID NO: 7 - DNA, 15 bp, synthetic construct
  source 1..15: mol_type = other DNA
ggttggtgtggttgg

SEQ ID NO: 8 - DNA, 17 bp, synthetic construct
  source 1..17: mol_type = other DNA
agtccgtggtaggggca

SEQ ID NO: 9 - DNA, 14 bp, synthetic construct
  source 1..14: mol_type = other DNA
acctgggggagtat

SEQ ID NO: 10 - DNA, 13 bp, synthetic construct
  source 1..13: mol_type = other DNA
tgcggaggaaggt

SEQ ID NO: 11 - DNA, 13 bp, synthetic construct
  source 1..13: mol_type = other DNA
acctggggagtat

SEQ ID NO: 12 - DNA, 13 bp, synthetic construct
  source 1..13: mol_type = other DNA
tgcgcaggaaggt

SEQ ID NO: 13 - DNA, 21 bp, synthetic construct
  source 1..21: mol_type = other DNA
attatagcggaggaaagtatt

SEQ ID NO: 14 - DNA, 21 bp, synthetic construct
  source 1..21: mol_type = other DNA
agtacctggggagtatatact

SEQ ID NO: 15 - DNA, 14 bp, synthetic construct
  source 1..14: mol_type = other DNA
acctgggggagtat

SEQ ID NO: 16 - DNA, 13 bp, synthetic construct
  source 1..13: mol_type = other DNA
tgcggaggaaggt

SEQ ID NO: 17 - DNA, 14 bp, synthetic construct
  source 1..14: mol_type = other DNA
atgcggaggaaggt

SEQ ID NO: 18 - DNA, 19 bp, synthetic construct
  source 1..19: mol_type = other DNA
agagaacctgggggagtat

SEQ ID NO: 19 - DNA, 17 bp, synthetic construct
  source 1..17: mol_type = other DNA
tgcggaggaaggttgag

SEQ ID NO: 20 - DNA, 15 bp, synthetic construct
  source 1..15: mol_type = other DNA
tacctgggggagtat

SEQ ID NO: 21 - DNA, 14 bp, synthetic construct
  source 1..14: mol_type = other DNA
ttgcggaggaaggt

SEQ ID NO: 22 - DNA, 40 bp, synthetic construct
  source 1..40: mol_type = other DNA
catctcttctccgagccggtcgaaatattggaggaagctc

SEQ ID NO: 23 - DNA, 22 bp, synthetic construct
  source 1..22: mol_type = other DNA
gagctggaggaaaaagtgacaa

SEQ ID NO: 24 - DNA, 27 bp, synthetic construct
  source 1..27: mol_type = other DNA
ttcgttcttcaatgaagtgggacgaca

SEQ ID NO: 25 - DNA, 16 bp, synthetic construct
  source 1..16: mol_type = other DNA
gggagtcaagaacgaa

SEQ ID NO: 26 - DNA, 13 bp, synthetic construct
  source 1..13: mol_type = other DNA
gggagtcaagaac

SEQ ID NO: 27 - DNA, 24 bp, synthetic construct
  source 1..24: mol_type = other DNA
gttcttcaatgaagtgggacgaca

SEQ ID NO: 28 - DNA, 24 bp, synthetic construct
  source 1..24: mol_type = other DNA
gttcttcaatgaagtgggacgaca

SEQ ID NO: 29 - DNA, 24 bp, synthetic construct
  source 1..24: mol_type = other DNA
gtccttcaacgaagtgggacgaca

SEQ ID NO: 30 - DNA, 40 bp, synthetic construct
  source 1..40: mol_type = other DNA
ctccttcaacgaagtgggttccttcaacgaagtgggtctc

SEQ ID NO: 31 - DNA, 18 bp, synthetic construct
  source 1..18: mol_type = other DNA
gagacaaggaacaaggag

SEQ ID NO: 32 - DNA, 39 bp, synthetic construct
  source 1..39: mol_type = other DNA
gtccactaacgaagttgtcttccactaacgaagttgtcc

SEQ ID NO: 33 - DNA, 18 bp, synthetic construct
  source 1..18: mol_type = other DNA
ggacgtggagacgtggac

SEQ ID NO: 34 - protein, 24 aa, synthetic construct
CGGCUGGGGCGAUACCAGCCGAAA

SEQ ID NO: 35 - protein, 28 aa, synthetic construct
CCAGCCGAAAGGCCCUUGGCACGUCGGG

SEQ ID NO: 36 - protein, 20 aa, synthetic construct
AAAGGCGAUACCAGCCGAAA

SEQ ID NO: 37 - protein, 20 aa, synthetic construct
GGCCCUUGGCAGCGUCAAAA

SEQ ID NO: 38 - DNA, 28 bp, synthetic construct
  source 1..28: mol_type = other DNA
tcgtgggacgacattcgttcttcaatga

SEQ ID NO: 39 - DNA, 15 bp, synthetic construct
  source 1..15: mol_type = other DNA
ggagtcaagaacgaa

SEQ ID NO: 40 - DNA, 24 bp, synthetic construct
  source 1..24: mol_type = other DNA
gttcttcaatgaagtgggacgaca

SEQ ID NO: 41 - DNA, 22 bp, synthetic construct
  source 1..22: mol_type = other DNA
catctcttctccgagccggtcg

SEQ ID NO: 42 - DNA, 12 bp, synthetic construct
  source 1..12: mol_type = other DNA
aaatagtgggtg

SEQ ID NO: 43 - DNA, 22 bp, synthetic construct
  source 1..22: mol_type = other DNA
catctcttctccgagccggtcg

SEQ ID NO: 44 - DNA, 27 bp, synthetic construct
  source 1..27: mol_type = other DNA
aaatagttggtcgctggggggctgacc

SEQ ID NO: 45 - DNA, 30 bp, synthetic construct
  source 1..30: mol_type = other DNA
atctgtcgagtggttacaccatgttcgtca

SEQ ID NO: 46 - DNA, 27 bp, synthetic construct
  source 1..27: mol_type = other DNA
tcttgcagcgattaacaactgatgcta

SEQ ID NO: 47 - DNA, 33 bp, synthetic construct
  source 1..33: mol_type = other DNA
tcaacatcagtcggtcgaaatagtgagtcgctc

SEQ ID NO: 48 - DNA, 27 bp, synthetic construct
  source 1..27: mol_type = other DNA
catctcttctccgagcctgataagcta

SEQ ID NO: 49 - DNA, 34 bp, synthetic construct
  source 1..34: mol_type = other DNA
acaggcggccttaactgtagttgggtagggcggg

SEQ ID NO: 50 - DNA, 22 bp, synthetic construct
  source 1..22: mol_type = other DNA
tgggtctggtgaaattgctgcc

SEQ ID NO: 51 - protein, 44 aa, synthetic construct
ACUGUUGAGUAGAGUGUGAGCUCCGCAGTGGCCCATACCCATGC

SEQ ID NO: 52 - protein, 20 aa, synthetic construct
UGGUGAAGGACGGGUCCAGU

SEQ ID NO: 53 - protein, 28 aa, synthetic construct
CAGAGAGACGGUCGGGUCCACAGUUUCC

SEQ ID NO: 54 - protein, 33 aa, synthetic construct
GGAAACUGUGUCGAGUAGAGUGUGGGCUCUCUG

(19) United States
(12) Patent Application Publication
(10) Pub. No.: US 2026/0210955 A1
(43) Pub. Date: Jul. 23, 2026

(54) SURFACE BOUND MULTI-BOND APTAMER SENSORS

(71) Applicant: University of Cincinnati, Cincinnati, OH (US)

(72) Inventor: Jason Heikenfeld, Cincinnati, OH (US)

(21) Appl. No.: 19/367,343

(22) Filed: Oct. 23, 2025

Related U.S. Application Data
(63) Continuation-in-part of application No. PCT/US2024/026672, filed on Apr. 26, 2024.
(60) Provisional application No. 63/462,075, filed on Apr. 26, 2023.
(60) Provisional application No. 63/711,818, filed on Oct. 25, 2024.

(51) Int. Cl. G01N 33/543; G01N 33/53

(52) U.S. Cl. CPC G01N 33/5438; G01N 33/5308; G01N 33/54373; G01N 2333/4737; G01N 2333/5412; G01N 2333/58; G01N 2333/62

(57) ABSTRACT

A device for detecting or measuring at least one large analyte in a sample fluid is provided. The device includes at least one substrate and a plurality of multi-bond aptamers capable of binding to the analyte. The aptamers are physically bound to the substrate and include at least one tag capable of providing a signal. Also, the multi-bond aptamer has a change in geometry when the multi-bond aptamer binds to the analyte. Further, the tag has a resulting change in signal resulting from the analyte binding to the multi-bond aptamer and associated change in geometry.

### CROSS-REFERENCE TO RELATED APPLICATIONS

This application is filed under 35 U.S.C. 111(a) as a bypass continuation-in-part application claiming priority to International Application No. PCT/US2024/026672 filed Apr. 26, 2024, which claims priority to, and benefit of the filing date of, U.S. Provisional Application Ser. No. 63/462,075, filed Apr. 26, 2023, the disclosures of which are hereby incorporated by reference herein in their entireties. The present bypass continuation-in-part application also claims priority to, and the benefit of the filing date of, U.S. Provisional Application Ser. No. 63/711,818, filed Oct. 25, 2024, the disclosure of which is hereby incorporated by reference herein in its entirety.

### INCORPORATION BY REFERENCE STATEMENT

The Sequence Listing XML file, identified as “23097,” having a file size of 48,460 bytes and created on Oct. 21, 2025, is incorporated by reference in its entirety as part of this application.

### FIELD OF THE INVENTION

This invention relates generally to aptamer sensors.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Electrochemical aptamer sensors can identify the presence and/or concentration of an analyte of interest via the use of an aptamer sequence that specifically binds to the analyte of interest. These sensors include aptamers attached to an electrode, wherein each of the aptamers has a redox active molecule (redox tag) attached thereto. The redox couple can transfer electrical charge to or from the electrode. When an analyte binds to the aptamer, the aptamer changes shape, bringing the redox couple closer to or further from, on average, the electrode. This results in a measurable change in electrical current that can be translated to a measure of concentration of the analyte. This same change in aptamer movement can also be translated into an optical measurement for an aptamer with optical tags such as fluorescent molecules and fluorescence quenchers. Aptamers are an example of an affinity-based biosensor.

A major unresolved challenge for aptamer sensors and other affinity-based biosensors is the limit of detection and magnitude of sensor response when larger aptamers are required for binding to the analyte, especially for measuring larger analytes such a peptide hormones and proteins. In fact, looking at existing demonstrations of electrochemical sensors for proteins you find them often limited to nM to μM detection ranges with <50% change in sensor response, which is insufficient for situations where most large analytes are in concentrations of pM to nM and far short of the 100-200% sensor responses that can be achieved for small molecule aptamer-based sensors. Aptamers can have improved binding affinity to the analyte of interest and therefore a lower limit of detection and a stronger sensor response using techniques such as purposeful mutations, larger aptamer sizes/lengths than exist in aptamer selection libraries, non-native base pairs, aptamer modification, non-DNA portions of an aptamer, and other methods. However, the length of such aptamers can be problematic not only in sensor response and sensor accuracy, but also in degradation by nucleases. Also, unfavorable secondary structures can interact with other solutes in complex media such as interstitial fluid and therefore reducing specificity (increasing interference). Novel approaches are therefore needed for electrochemical aptamer sensors which eliminate the drawbacks of limited binding affinity, enzyme attack, sensor response, accuracy, and specificity.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

One aspect of the present invention is directed to a device for detecting or measuring at least one large analyte in a sample fluid. The device includes at least one substrate; a sample fluid comprising the analyte; and a plurality of multi-bond aptamers capable of binding to the analyte, wherein the aptamers are physically bound to the substrate. The multi-bond aptamers include at least one tag capable of providing a signal. Also, the multi-bond aptamer has a change in geometry of the multi-bond aptamer when the multi-bond aptamer binds to the analyte. In addition, the at least one tag has a resulting change in signal resulting from the analyte binding to the multi-bond aptamer and associated change in geometry.

In one embodiment, the multi-bond aptamer has two or more binding portions. In another embodiment, the tag is a redox tag. In one embodiment, the substrate is an electrode. In another embodiment, the tag is an optical tag. In one embodiment, the substrate is an optical waveguide. In another embodiment, the plurality of multi-bond aptamers further comprise at least one first binding portion and at least one second binding portion. In one embodiment, one or more of the plurality of multi-bond aptamers further comprise at least one flexible linking portion.

In another embodiment, the first binding portion and the second binding portion are separated by at least one connection of molecules. Further, the at least one connection of molecules comprises at least one of a) molecules not binding to the analyte during binding of the multi-bond aptamer to the target analyte; b) at least one molecule that attaches at least the first binding portion and at least the second binding portion to the substrate; c) a separation resulting from the first portion and the second portion having independent binding chemistry linkages to the substrate on which they are bound.

In one embodiment, the first binding portion and the second binding portion are bound to the substrate and are further bound to each other. In another embodiment, the first binding portion and the second binding portion are bound to the substrate and are further bound to each other by base-pair matching between a plurality of base pairs. In one embodiment, at least one of the first binding portion or the second binding portion form a stem loop in absence of analyte binding to the multi-bond aptamer. In another embodiment, the device has a sensor response, and the sensor response is a percentage selected from the group consisting of >50%, >100%, and >200%.

Another aspect of the present invention is directed to a device for detecting or measuring at least one large analyte in a sample fluid. The device includes at least one substrate and a plurality of aptamers capable of binding to the analyte. The aptamers are physically bound to the substrate. The aptamers include at least one tag capable of providing a signal. Further, the aptamer has a change in geometry of the aptamer when the aptamer binds to the analyte. Also, the at least one tag has a resulting change in signal resulting from the analyte binding to the aptamer and associated change in geometry. In addition, the aptamers further include at least a first portion with at least one tag that does not bind to the analyte, and at least a second portion which does bind to the analyte.

In one embodiment, the first portion has a weaker binding affinity than the second portion and the first portion is shorter in length than the second portion. In another embodiment, the first portion has at least one tag and forms a stem loop in absence of binding of analyte to the second portion. In one embodiment, the tag is a redox tag. In another embodiment, the substrate is an electrode.

In one embodiment, the tag is an optical tag. In another embodiment, the substrate is an optical waveguide. In one embodiment, the device also includes at least one flexible linking portion between the first portion and the second portion.

In another embodiment, the first portion and the second portion are separated by at least one connection of molecules. The at least one connection of molecules comprises at least one of a) molecules not binding to the analyte during binding target analyte; b) at least one molecule that attaches at least the first portion and at least the second portion to the substrate; c) a separation resulting from the first portion and the second portion having independent binding chemistry linkages to the substrate on which they are bound.

In one embodiment, at least the first portion and at least the second portion are bound to the substrate and are further bound to each other. In another embodiment, at least the first portion and at least the second portion are bound to the substrate and are further bound to each other by base-pair matching between a plurality of base pairs. In one embodiment, the device has a sensor response, and the sensor response is a percentage selected from the group consisting of >50%, >100%, or >200%. In another embodiment, the multi-bond aptamer is further comprised of at least a first portion and a least a second portion, and further, wherein when the first portion binds the analyte the second portion binds a least in part to the first portion. In one embodiment, when the first portion binds the analyte the second portion binds a least in part to the first portion.

Another aspect of the present invention is directed to a method for detecting or measuring at least one large analyte in a sample fluid. The method involves exposing the sample fluid to the device described above and detecting or measuring the amount of the large analyte using data regarding changes in one or more aptamer parameters. In one embodiment, the sample fluid is exposed to the device by placing the device in direct contact with the sample fluid or placing the device in fluid communication with the sample fluid.

Another aspect of the present invention is directed to a device for detecting or measuring at least one analyte in a sample fluid, wherein the device includes at least one substrate, and a plurality of aptamer pairs, each aptamer pair comprising a first aptamer and a second aptamer, wherein at least the first aptamer or second aptamer of each of the plurality of aptamer pairs are bound to the substrate. In this aspect, the first aptamer includes at least one tag, and the second aptamer is capable of binding the at least one analyte. In each aptamer pair, the first aptamer and second aptamer are sufficiently complementary to hybridize to one another in the absence of analyte binding to the second aptamer, and signal from the tag to the substrate occurs through the hybridized first and second aptamers. Finally, when analyte is present and binds to the second aptamer, the first aptamer and second aptamer separate to a degree that reduces or eliminates signal from the tag to the substrate. This change in signal can be detected.

Another aspect of the present invention is directed to a method for detecting or measuring at least one analyte in a sample fluid, the method comprising exposing a sample fluid to the device including a plurality of aptamer pairs, and detecting or measuring the amount of the analyte using data regarding changes in one or more parameters of the first and/or second aptamers of the aptamer pairs.

### BRIEF DESCRIPTION OF THE DRAWINGS

The objects and advantages of the disclosed invention will be further appreciated in light of the following detailed descriptions and drawings in which:

FIG. 1 is a schematic of a conventional prior art sensor device.

FIG. 2 is a schematic of a conventional prior art sensor device.

FIG. 3 is a schematic of a device of an embodiment of the present invention with a multi-bond aptamer.

FIG. 4 is a schematic of a device of an embodiment of the present invention with a multi-bond aptamer.

FIG. 5 is a schematic of a device of an embodiment of the present invention with a multi-bond aptamer.

FIG. 6 is a schematic of a device of an embodiment of the present invention with a multi-bond aptamer.

FIG. 7 is a schematic of a device of an embodiment of the present invention with a multi-bond aptamer.

FIG. 8 is a schematic of a device of an embodiment of the present invention with a multi-bond aptamer.

FIG. 9 is a schematic of a device of an embodiment of the present invention with a multi-bond aptamer.

FIG. 10 is an illustration of Thrombin indicating the large variety of binding sites.

FIG. 11 is a list of additional FBPMA and SBPMA that may be useful in the present invention. Either Fragment 1 or Fragment 2 can be the FBPMA or SBPMA.

FIG. 12 is a list of additional FBPMA and SBPMA that may be useful in the present invention. Either Fragment 1 or Fragment 2 can be the FBPMA or SBPMA.

FIG. 13 is a schematic of a device of an embodiment of the present invention having a plurality of aptamer pairs, each aptamer pair including a first aptamer and a second aptamer that hybridize to one another in the absence of analyte.

### DEFINITIONS

As used herein, the term “about,” when referring to a value or to an amount of mass, weight, time, volume, pH, size, concentration or percentage is meant to encompass variations of ±20% in some embodiments, ±10% in some embodiments, ±5% in some embodiments, ±1% in some embodiments, ±0.5% in some embodiments, and ±0.1% in some embodiments from the specified amount, as such variations are appropriate to perform the disclosed method.

As used herein, the term “analyte” means any solute in a solution or fluid which can be measured using a sensor. Analytes can be small molecules, proteins, peptides, electrolytes, acids, bases, antibodies, molecules with small molecules bound to them, DNA, RNA, drugs, chemicals, pollutants, or other solutes in a solution or fluid.

As used herein, the term “aptamer” means a molecule that undergoes a conformation or binding change as an analyte binds to the molecule, and which satisfies the general operating principles of the sensing method as described herein. Such molecules are, e.g., natural or modified DNA, RNA, or XNA oligonucleotide sequences, spiegelmers, peptide aptamers, and affimers. Modifications may include substituting unnatural nucleic acid bases for natural bases within the aptamer sequence, replacing natural sequences with unnatural sequences, or other suitable modifications that improve sensor function, but which behave analogous to traditional aptamers.

As used herein, the term “change in electron transfer” means a redox molecule whose electron transfer with an electrode has changed in a measurable manner. This change in electron transfer can, for example, originate from availability for electron transfer, distance from an electrode, diffusion rate to or from an electrode, a shift or increase or decrease in electrochemical activity of the redox molecule, or any other embodiment as taught herein that results in a measurable change in electron transfer between the redox molecule and the electrode.

As used herein, the term “continuous sensing” means the device records a plurality of readings over time. As used herein, “continuous sensing” with a “continuous sensor” means a sensor that changes in response to changing concentration of at least one solute in a solution such as an analyte. Similarly, as used herein, “continuous monitoring” means the capability of a device to provide multiple measurements of an analyte over time.

As used herein, a “device” comprises at least one sensor based on at least one aptamer and at least one sample solution. Devices can sense multiple samples and be in multiple configurations such as a device to measure a pin-prick of blood, or a microneedle or in-dwelling sensor needle to measure interstitial fluid, or a device to measure saliva, tears, sweat, or urine sensor, or a device to measure water pollutants or food processing solutes, or other devices which measure at least one analyte found in a sample solution.

As used herein, the term “electrode” means any material that is electrically conductive such as gold, platinum, nickel, silicon, conductive liquid infused materials such as ionic liquids, PEDOT:PSS, conductive oxides, carbon, boron-doped diamond, nanotubes or nanowire meshes, or other suitable electrically conducting materials.

As used herein, the term “fluid communication” means that the elements are coupled together with an appropriate lumen, supply passage, line or other means to permit the passage of fluid or connection to fluid (interstitial fluid, blood, water, saline, etc.) therebetween.

As used herein, the term “large analyte” means an analyte with >1 kDa of molecular weight and in most cases with be >3 kDa such as insulin, BNP or NT-proBNP, or even >10 kDa such as monomeric C-reactive protein, IL-6 or other suitable analytes. As used herein, the term “small analyte” means an analyte that is less than or equal to 1 kDa.

As used herein, the term “multi-bond aptamer” means two or more portions of an aptamer or two or more aptamers, whose connection to a substrate is physically attached between the two or more portions or aptamers, and where the connection to the substrate may be a single connection or multiple connections that are bound together and therefore similar to a single connection to the substrate, and the two or more portions or aptamers bind to two or more different regions of an analyte. In most cases the aptamers will have a difference in their sequence but for analytes with a high degree of chemical and geometrical symmetry the aptamers can be the same and be referred to as a symmetric multi-bond aptamer.

As used herein, the term “non-native” portion of an aptamer is comprised of a material other than DNA. For example, phosphoramidite can be used for conjugation of oligonucleotides with biomolecules, using for example polyethylene glycol phosphoramidite.

As used herein, the term “optical tag” means fluorescent, quencher, and fluorescence resonance energy transfer, or other optical tags, such as those used in molecular beacon laboratory assays. Examples of fluorescent tags include 6-FAM (carboxylflourescien), JOE, TET, HEX, and examples of quenchers include black-hole quenchers, DABCYL. These tags may also be referred to as “optical tags” more generally, as there are multiple types of optical emission beyond fluorescence such as phosphorescence, and because other optical properties such as optical absorbance magnitude or peak wavelength for optical absorption can also be measurable aspects of the tags.

As used herein, the term “optical waveguide” means a material that confines light by total internal reflection. For example, a waveguide could be glass or plastic with a smooth surface surrounded by a material with lower refractive index than the waveguide.

As used herein, the term “protective layer” means a homogeneous or heterogeneous layer of material or of one or more types of molecules on an electrode or other aptamer sensor surface which reduce background signal and interference, and which may promote proper freedom of movement for the aptamer which is required for creating a measurable response to analyte concentration. Protective layers may be formed, for example, on electrode, waveguides, or other suitable surfaces.

As used herein, the term “redox tag” or “redox molecule” means any species such as small or large molecules with a redox active portion that when brought adjacent to an electrode can reversibly transfer at least one electron with the electrode. Redox tag or molecule examples include methylene blue, ferrocene, quinones, or other suitable species that satisfy the definition of a redox tag or molecule. In some cases, a redox tag or molecule is referred to as a redox mediator. Redox tags or molecules may also exchange electrons or change in behavior when brought into proximity with other redox tags or molecules. Redox tags can be tagged at the end of an aptamer or internally along the aptamer using for example thymine base modification, referred to as ‘distal tagging’ and ‘internal tagging”, respectively.

As used herein, “redox tag current” means the amplitude of the faradaic redox tag peak current minus the background current amplitude outside the redox peak in a given voltammetric scan.

As used herein, the term “sensing monolayer” means at least a plurality of aptamers on a sensor surface, which may also include a plurality of molecules or mixtures of molecules that form a protective layer.

As used herein, “sensor response” is the change in sensor signal due to binding of the target analyte to the aptamer, also known as signal gain, which can either increase or decrease based on the aptamer and the time scale of the measurement. Sensor response may also be applied to alternative measures such as amperometry or chronoamperometry or fluorescence lifetime, or other approaches. As used herein “change in sensor response” is the percentage change in the sensor response in response to increasing analyte concentration compared to sensor response if no analyte were present. For example, if the peak redox tag current was 1 μA with no analyte present and adding analyte caused the peak redox tag current to be 0.5 or 1.5 μA then the change in sensor response would be −50% sensor off response or +50% sensor on response respectively.

As used herein, the term “tag” means a molecule attached to an aptamer with at least one measurable change in the behavoir of the molecule caused by binding of the aptamer to an analyte. For example, in response to analyte binding to the aptamer the tag could have more or less redox electron transfer, or more or less fluorescence.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers” specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain embodiments of the disclosed invention show sensors as simple individual elements. It is understood that many sensors require two or more electrodes, reference electrodes, or additional supporting technology or features which are not captured in the description herein. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a reference or counter electrode, a battery, antenna, adhesive), and for purposes of brevity and focus on inventive aspects, such components may not be explicitly shown in the diagrams or described in the embodiments of the disclosed invention. All ranges of parameters disclosed herein include the endpoints of the ranges.

In one embodiment, the device of the present invention is used to detect and/or measure at least one large analyte in a sample fluid. The device includes at least one substrate; a sample fluid comprising the analyte; and a plurality of multi-bond aptamers capable of binding to the analyte, wherein the aptamers are physically bound to the substrate. The multi-bond aptamers include at least one tag capable of providing a signal. Also, the multi-bond aptamer has a change in geometry of the multi-bond aptamer when the multi-bond aptamer binds to the analyte. In addition, the at least one tag has a resulting change in signal resulting from the analyte binding to the multi-bond aptamer and associated change in geometry.

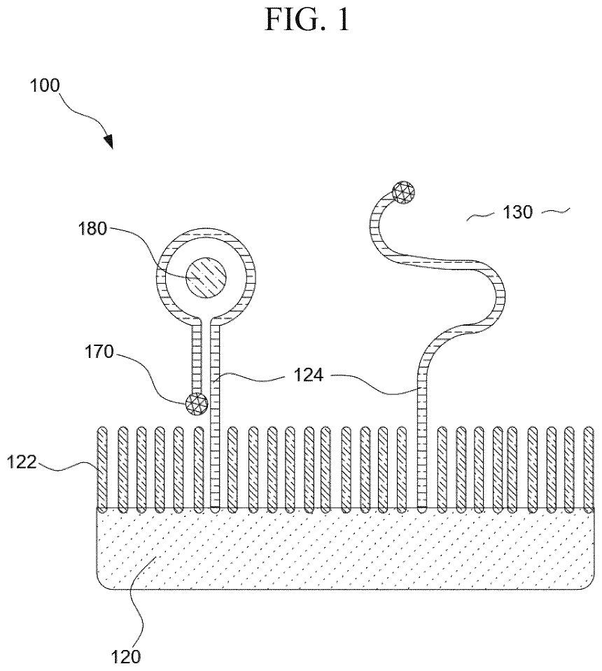

With reference to FIG. 1A, a conventional prior art sensor device 100 as placed initially in a sample fluid 130 such as interstitial fluid is shown, comprising: at least one working electrode 120 such as gold, carbon, or other suitable electrode material; at least one blocking or protective layer 122 of a plurality of molecules such as mercaptohexanol or mercaptoctanol that are thiol bonded to the electrode, or other suitable molecules depending on application and on the choice of electrode 120 material; at least one aptamer 124 that is linked to the gold 120 via a thiol bond or other suitable bond with a 6 methyl (carbon) or 8 methyl (carbon) linker to an electrode 120 or to a protective layer 122. The aptamer 124 is responsive to binding to an analyte 180 and which contains a redox tag 170 such as methylene blue. In the generic example taught for FIG. 1, the aptamer 124 is a simple stem loop (hairpin) aptamer where analyte 180 binding causes the stem loop to form and the redox tag current measured from the redox tag 170 to increase, as measured using square wave voltammetry or other suitable technique. The sensor can also have more than one stem loop, for example such as the cocaine aptamer which has >100% sensor response to cocaine as taught in White et al. Langmuir 2008, 24, 18, 10513-10518. In the absence of analyte 180 binding to the aptamer 124, the stem loop is broken and the redox current would decrease. Thus, a measurement of electrical current can be used to interpret changes in the concentration of the analyte 180. The majority of electrochemical aptamer sensors use ‘signal ON’ motifs like that illustrated in FIG. 1 which produce large sensor responses of >100% by leveraging a stem loop or other stable configuration which brings the redox tag 170 close to the electrode 120 in a stable geometry with significantly increased redox tag current. These ‘signal ON’ geometries can use a single binding domain of the aptamer 124 to the analyte 180 because the analyte is a small molecule and because the small molecule is at high concentrations such that single binding domain can be sufficient. Making a similar sensor for a larger peptide or protein or other analyte such as insulin, BNP, c-reactive protein, IL-6 or other analytes is much more difficult because they are at much lower concentrations (pM to nM range) and because they are so large that a single binding domain will have difficulty in many cases binding to the analyte with a binding affinity that is close to the physiologically expected concentrations in a biofluid such as interstitial fluid.

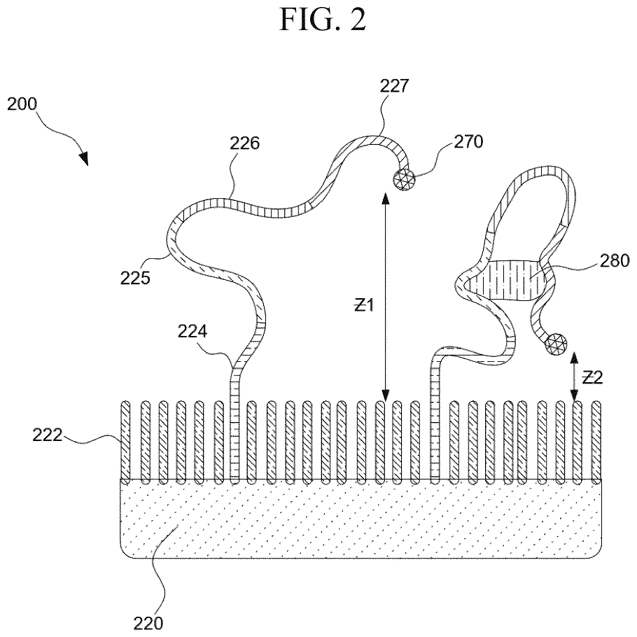

With reference to FIG. 2, where like numerals refer to like features a device 200 having an electrode 220 and a protective layer 222 utilizes an aptamer comprised of elements 224, 225, 226, and 227. Element 224 can be, for example, a substrate linking portion of an aptamer. Element 225 can be, for example, a first binding portion of an aptamer. Element 226 can be, for example, a flexible linking portion of an aptamer, and element 227 can be a second binding portion of an aptamer which includes a distally positioned redox tag 270 such as methylene blue. The specific geometries, lengths, and other features of FIG. 2 should only be limited as specifically specified herein, as there are multiple stable, semi-stable, and freely moving geometries possible for aptamer design. FIG. 2 will now be taught in greater detail. When Element 224 is the substrate linking portion of the aptamer it can be a variety of sequences or materials as used in conventional electrochemical aptamer-based sensors and its length and flexibility optimized for a given analyte 280. When Element 225 is a first binding portion of the aptamer, it can be a variety of sequences or materials as used for example in aptamers or other linkers used in biosensor chemistry, including for example a chain of thymine bases, or for example non-native chains of polyethylene glycol. When Element 225 is a first binding portion of an aptamer, and Element 227 is a second binding portion of an aptamer, they are chosen or designed to bind to distinct portions of the large analyte 280 inducing a change in availability of the redox tag 270 for electron transfer with electrode 220. This change in availability, for simplicity of illustration, is depicted as a change in distance Z1, Z2, as labeled in FIG. 2, and the exact nature of change in electron transfer can of course be more complicated as understood by those skilled in the art of electrochemical aptamer-based sensors. The device 200, could use a combination of first and second portions to achieve a lower limit of detection and overall binding affinity than what an individual binding portion could achieve alone. For example, insulin is a high-value large analyte target for which it is difficult to make a robust binding aptamer with a single binding site (e.g. FIG. 1) that measured insulin at physiological concentrations. Two or more binding sites (a plurality) and two or more binding portions of the aptamer in combination lowers the limit of detection by increasing the overall binding affinity by, in alternate embodiments, at least 3×, 10×, 30×, 100×, or more.

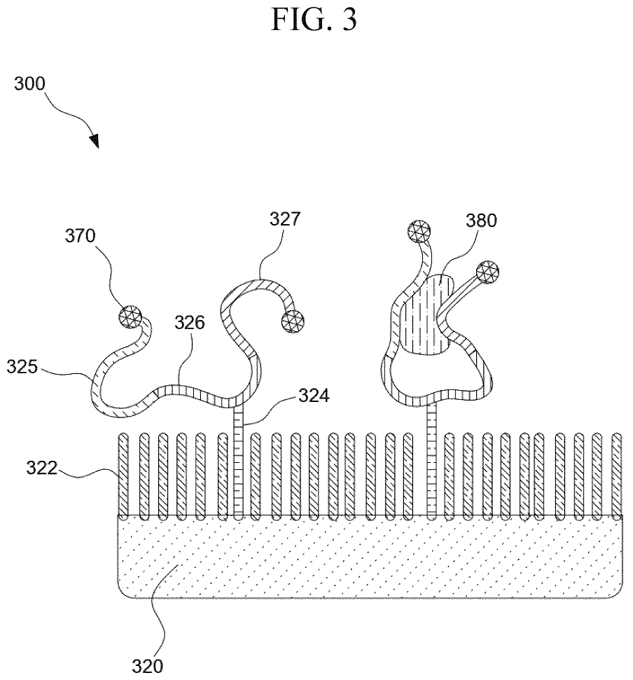

With reference to FIG. 3, where like numerals refer to like features, in an embodiment of the present invention a device 300 uses multi-bond aptamers for detection and has via attachment a first binding portion of a multi-bond aptamer (FBPMA) 325 and a second binding portion of a multi-bond aptamer (SBPMA) 327 that are branched and connected by at least one point between the FBPMA and SBPMA with a flexible linking portion of a multi-bond aptamer (FLPMA) 326 that is connected to a substrate-linking portion of a multi-bond aptamer (SLPMA) 324 using one of numerous methods to covalently bond to an aptamer (such as used to covalently label aptamers) including click chemistry such as taught by Kocker et al in ‘Covalent labeling of nucleic acids’ Chem. Soc. Rev., 2020,49, 8749-8773. Examples include copper (I)-catalyzed Azide-Alkyne Cycloaddition (CuAAC) between a terminal alkyne and an azide, copper-free click reactions, like the Strain-Promoted Azide-Alkyne Cycloaddition (SPAAC) and the Inverse Electron-Demand Diels-Alder cycloaddition (IEDDA), tetrazine ligation—an IEDDA reaction between a tetrazine and an alkene, providing a linker attachment point or linker itself during solid-phase synthesis, enzyme labeling of reactive species that can attach the linker, or other suitable methods. The FBPMA and/or the SBPMA can be redox tagged with a tag such as methylene blue 370. The analyte 380 binding alters the availability of the redox tags 370 to exchange electrons with the electrode 320, and as illustrated in FIG. 3 the redox tags 370 are positioned further from the electrode 320 resulting in less redox current. An advantage of this configuration is the FBPMA and SBPMA can be optimally positioned together, which can be more difficult if a sensing monolayer of mixed aptamers were used (e.g. the FBPMA and SBPMA individually and randomly bound to the electrode 320). The present invention therefore includes a FBPMA and a SBPMA that are branched and connected to the electrode by at least one SLPMA attachment point between the FBPMA and SBPMA. The term “connected to the electrode” further includes any intervening materials between aptamers and electrode that effectively capture attachment as well (such as an intervening thin, porous silica layer and silane chemistry attachment to the silica). The device 300 therefore represents an embodiment having a “two or more binding portions” or a “plurality of binding portions” for a multi-bond aptamer where the binding portions are separated by at least one connection of molecules. In one embodiment, the “at least one connection of molecules” includes molecules not binding to the target analyte during binding of the aptamer to the target analyte. In an alternate embodiment, the FLPMA 326 can be removed (not shown), and the FBPMA 325 and SBPMA 327 are connected directly to each other at a point determined by connection to the SLPMA 324. This alternate embodiment of device 300 therefore represents an embodiment having “two or more binding portions” or a “plurality of binding portions” for a multi-bond aptamer where the binding portions separated by at least one connection of molecules. In this embodiment, the “at least one connection of molecules” includes at least one molecule that attaches the two or more binding portions to the substrate.

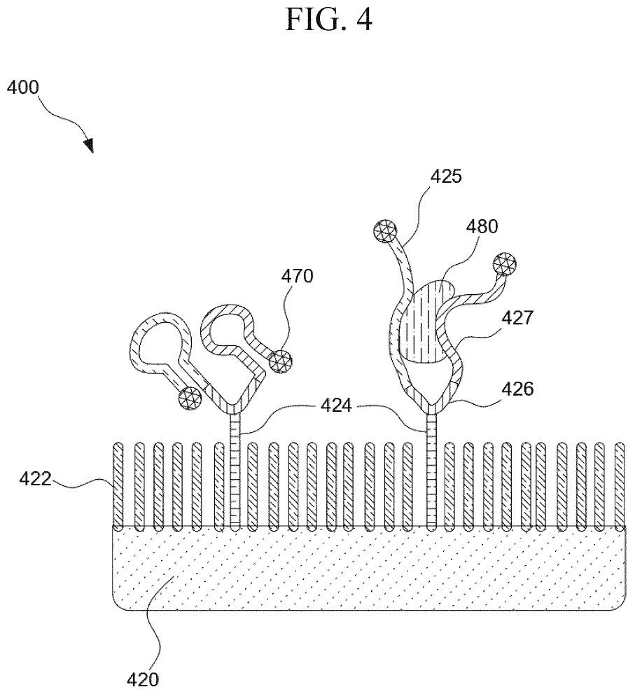

With reference to FIG. 4, where like numerals refer to like features such as blocking layer 422 and SLPMA 424, in an embodiment of the present invention a device 400 has multi-bond aptamers and has a FBPMA 425 and/or SBPMA 426 that form stem-loop or other structures that in the absence of analyte 480 binding cause an even greater redox-tag current by increasing availability for electron transfer with the electrode 420.

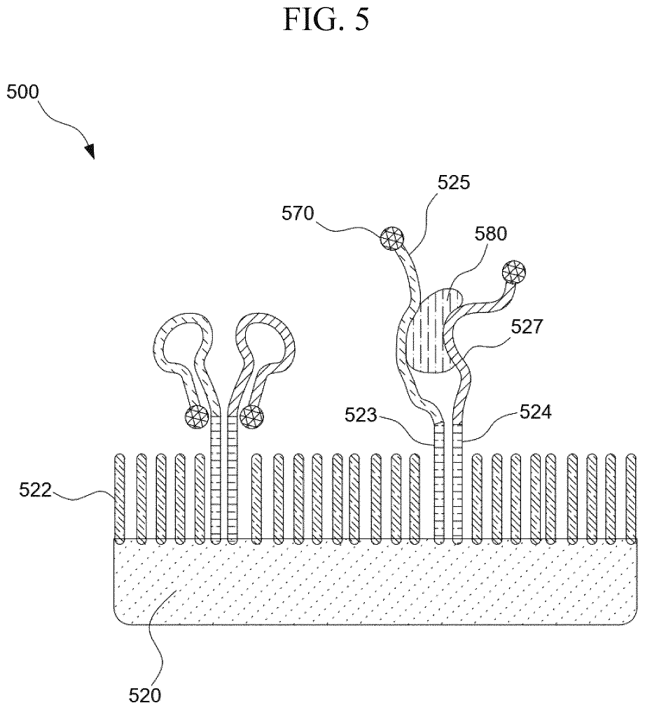

With reference to FIG. 5, where like numerals refer to like features, in an embodiment of the present invention a device 500 has multi-bond aptamers and captures a more organized spacing of FBPMA and SBPMA in an alternative way. The FBPMA 525 has a first SLPMA 523 while the SBPMA 527 has a second SLPMA 524 such that the FBPMA and SBPMA are individually bound to the electrode 520. To ensure FBPMA and SBPMA are optimally spaced, the first and second SLPMA's 524, 525 are comprised at least in part of complimentary base-sequences (such as 3, 4, 5, 6, 7 or more A-T or G-C base matches) or other chemical features such as repeat phenyl or alkane groups that rely on Vander Waals, pi stacking, or other suitable forces or chemical bonds, such that first and second SLPMA's 523 and 524 form a STEM such that during aptamer incubation and attachment onto the electrode 520 the first and second SLPMA's 524, 525 are immediately adjacent to one another. The present invention therefore includes a FBPMA with a first SLPMA and a SBPMA with a second SLPMA, and wherein the first and second SLPMA are both bound to the electrode and further bound to each other by base pair matching between a plurality of base pairs (such as GGCAGTG and CCGTCAC). The present invention therefore includes a FBPMA with a first SLPMA and a SBPMA with a second SLPMA, and wherein the first and second SLPMA are both bound to the electrode and further bound to each other. More generally, base pair matching is not required and other means may be used to associate the FBPMA and SBPMA so long as they achieve the same net effect of locally associating the FPMBA and SBPMA. This embodiment of device 500 therefore represents an embodiment having “two or more binding portions” or a “plurality of binding portions” for a multi-bond aptamer where the binding portions separated by at least one connection of molecules. In this embodiment, the “at least one connection of molecules” includes being separated by each having independent binding chemistry linkage to the substrate on which they are bound.

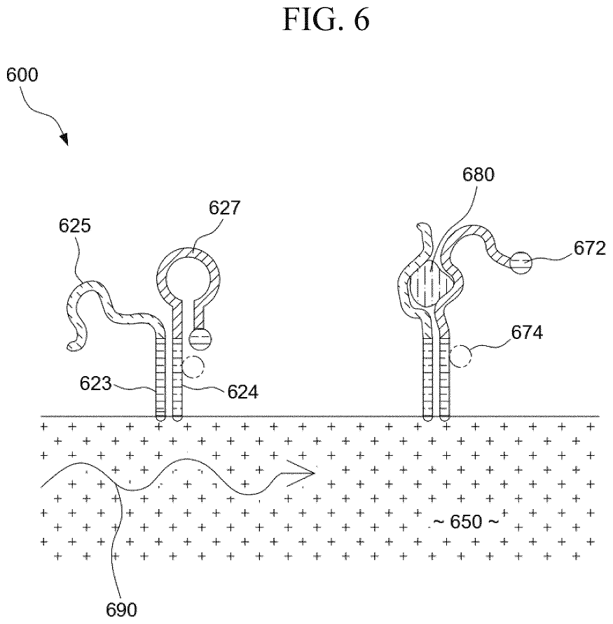

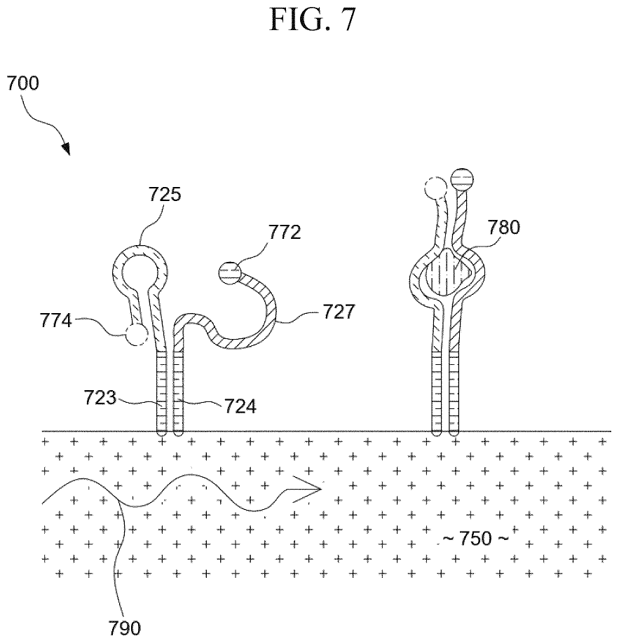

With reference to FIGS. 6 and 7 where like numerals refer to like features, in an embodiment of the present invention sensors with multi-bond aptamers may be optical in nature. Additional elements include optical waveguide 650 and light 690. The FPBA 625 and SBPMA 627 are specific to the analyte 680 such as a steroid hormone, a drug, a protein, etc. and for device 600 when the analyte and aptamer bind the fluorescent tag 672 is moved further away from a quencher tag 674 that quenches optical emission from tag 672. For device 700 analyte 780 binding causes tags 774, 772 to instead become closer. Because aptamers are within roughly a wavelength of light distance from an optical material such as a waveguide 650 they can be excited by light 690 through evanescent coupling or via light escaping waveguide 650 through refraction or scattering. Optical tags 672, 674 can be distally tagged at the ends of aptamers or internally tagged at FBPMA, SBPMA, FBPMA, or SLPMA. Optical tags 672, 674 can be a quencher and fluorophore, a donor and acceptor fluorophore pair for Fluorescence Resonance Energy Transfer signaling or other suitable optical tags. When tags 672, 674 are brought closer together, the fluorescence emission is quenched or experiences resonance transfer. There are a large number of examples for other types of fluorescent aptamer sensors, as taught for example in “Situma C, Moehring A J, Noor M A, Soper S A. Immobilized molecular beacons: a new strategy using UV-activated poly(methyl methacrylate) surfaces to provide large fluorescence sensitivities for reporting on molecular association events. Anal Biochem. 2007 Apr. 1; 363(1):35-45. doi: 10.1016/j.ab.2006.12.029. Epub 2006 Dec. 20. PMID: 17300739; PMCID: PMC2836515.” Another example is taught in “De Acha N, Elosúa C, Arregui FJ. Development of an Aptamer Based Luminescent Optical Fiber Sensor for the Continuous Monitoring of Hg²⁺ in Aqueous Media. Sensors (Basel). 2020 Apr. 22; 20(8):2372. doi: 10.3390/s20082372. PMID: 32331372; PMCID: PMC7219322.” The aptamers generally, but not always, require at least two tags (fluorescent and quencher tags) and a linker to bind the aptamer to the substrate, and any one of the tags or linkers can be attached at the 3′ end of the aptamer, the 5′ end of the aptamer, or at internal locations where the most common and widely utilized location is at an internal thymine site of the aptamer. The devices 600 and 700 may also utilize antifouling chemistries (not shown) such as silane bonded zwitter ions that are bound to the waveguide 650, 750. The present invention therefore includes a FBPMA and a SBPMA that are connected to at least one optical material by at least one SLPMA attachment point between the FBPMA and SBPMA. The present invention therefore includes a FBPMA with a first SLPMA and a SBPMA with as second SLPMA, and wherein the first and second SLPMA are both bound to an optical material and further bound to each other by base pair matching. The present invention therefore includes other embodiments taught for the present invention but applied to optical tagged sensing.

With further reference to FIGS. 4 and 5, and in embodiments of the present invention, at least one of the FBPMA or SBPMA or neither may form at least a partial STEM LOOP geometry. For example, in FIGS. 4 and 5 in the absence of the analyte binding, both the FBPMAs 425, 525 and SBPMAs 427, 527 form a STEM LOOP geometry which can cause a greater sensor response by predictably placing the tags 470, 570 in a fixed location without analyte binding to the multi-bond aptamer.

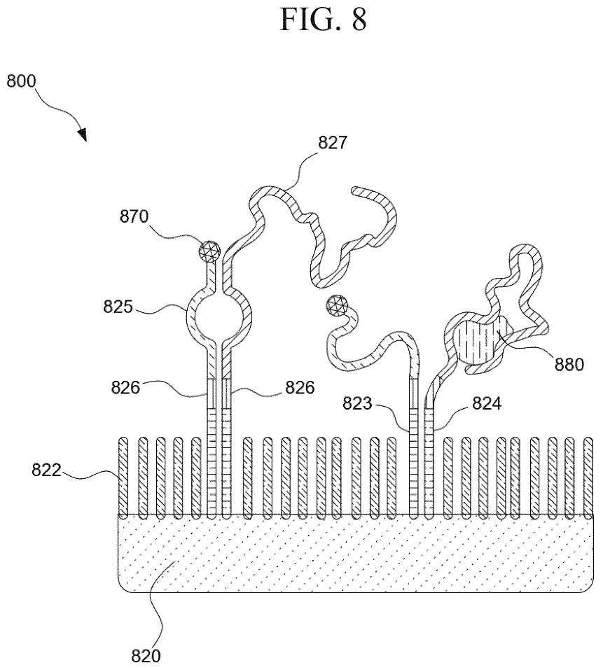

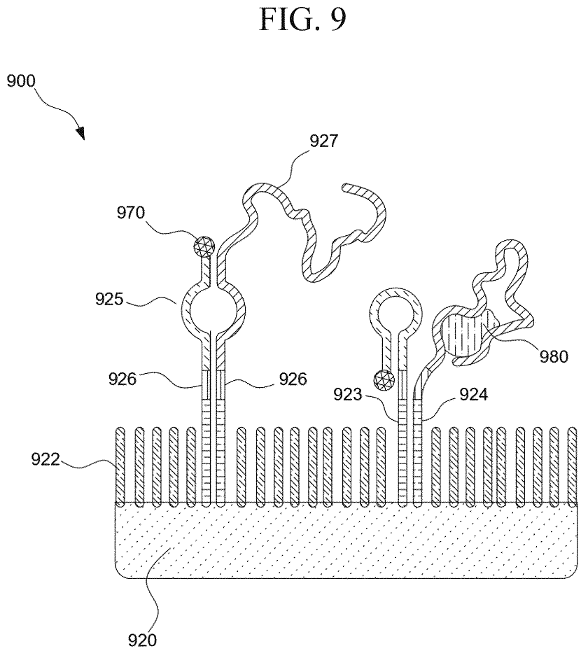

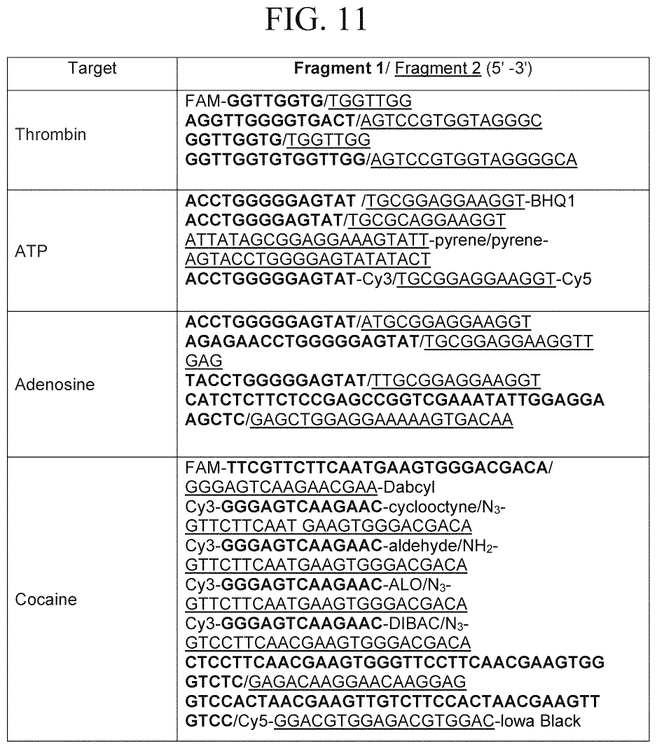

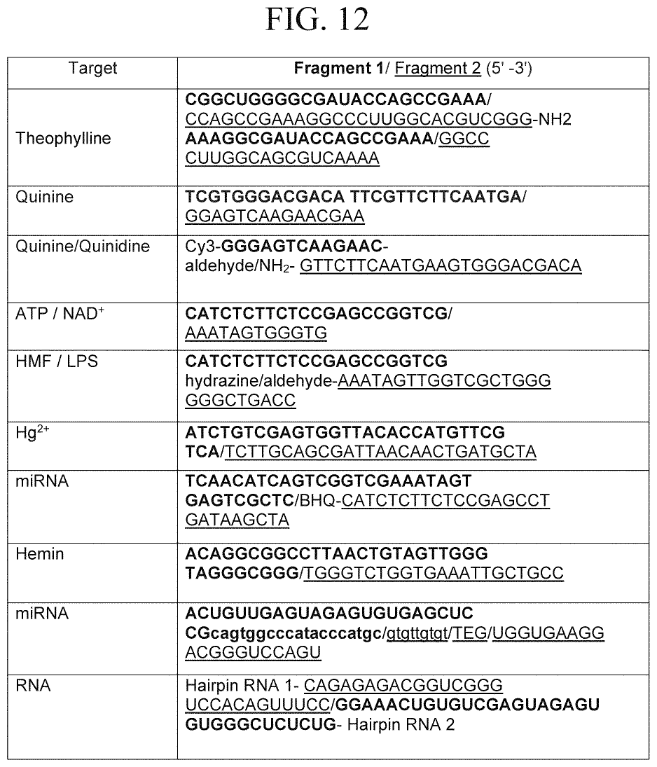

With reference to FIGS. 8 and 9 where like numerals refer to like features, an embodiment of the present invention is a multi-bond aptamer device 800 that can have only one binding portion of an aptamer that binds to the analyte. 823 and 824 provide linkage to the substrate 820 (electrode, waveguide, etc.) and elements 826 could be flexible portions as described previously or complimentary portions as describe previously for base-pair matching. For example, binding portion 827 is designed to bind to the analyte, while aptamer 825 is designed for optimal electrochemical (shown) or optical (not shown) signaling. Simply, some of the best binding aptamers for large analytes are like those demonstrated by SomaLogic (SOMAmers) and others and are in some cases too large to provide themselves a strong change in signal or sensor response. Instead, the device 800 breaks up the functioning of signaling and binding between a first portion of aptamer 825 and a second portion of aptamer 827. For example, in FIG. 9 the first portion has at least one tag that is signaling and forms a STEM LOOP in absence of the analyte binding to the second portion. Similarly, device 400 of FIG. 4 could be modified such that only aptamer 425 is signaling and aptamer 427 is not signaling. For example, aptamer 427 could be a somamer for IL-6, as demonstrated in the literature, which has a very strong binding affinity of 200 pM. Aptamer 425 could be one of the many other weaker binding aptamers for IL-6 demonstrated in the literature (or found separately via aptamer SELEX) but the aptamer is shorter and weaker binding. Because aptamer 427 would localize the analyte 480 by capturing it, it effectively increases the local concentration of target 480 such that the weaker binding aptamer 425 can then bind to it despite its weaker binding affinity. This alternate approach is desirable because when designing a smaller aptamer 425 for strong signaling you will often lose binding affinity and this example is a way to still use the weaker binding affinity in a sensor by assistance provided by aptamer 427 that has much stronger binding affinity. Therefore, the present invention may include a multi-bond aptamer with a FBPMA that is signaling (has a tag) and which independently has a weaker binding affinity than and which is shorter in length than a SBPMA. Several examples are shown in FIGS. 11 and 12, where generally the shorter portions of the aptamers have weaker binding affinities. The multi-bond aptamer may therefore have “two or portions” or a “plurality of portions” where at least one first portion includes at least one tag such as redox or optical tag, and where at least one second portion binds to the analyte, with the additional requirement that at least the first and second portion are separated by at least one connection of molecules, this at least one connection of molecules including at least one of: (1) molecules not binding to the analyte during binding target analyte; (2) at least one molecule that attaches at least the first portion and at least the second portion to the substrate; (3) a separation by at least the first portion and at least the second portion having independent binding chemistry linkage to the substrate on which they are bound.

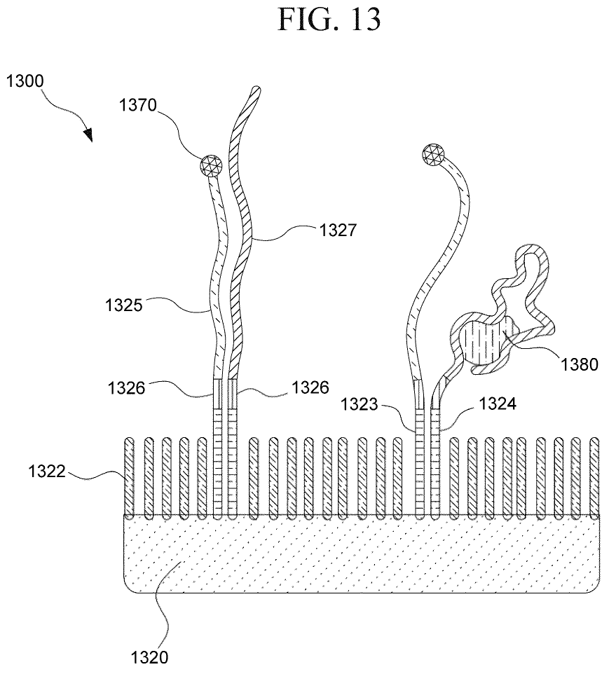

With further reference to FIGS. 8 and 9 and 13 where like numerals refer to like features, additional embodiments of the present invention are taught. Embodiments of the present invention may more generally include a first binding aptamer 827 that binds to the analyte and a second signaling aptamer 825 that is at least partially complimentary to the first binding aptamer 827. These first and second aptamers may also be fully hybridized or nearly fully hybridized as illustrated in FIG. 13 where aptamer 1325 is shorter in length than aptamer 1327. The first aptamer may for example form a stem-loop or other self-base-pairing configuration like that shown in FIG. 9 for aptamer 925. For example, the binding affinity for forming the stem-loop or other self-base-pairing configuration could be in the micromolar range, the hybridization between the first and second aptamers be in the nanomolar range, and the binding to the analyte 980 be in the picomolar range. Aptamer sequences may use sequences similar to those illustrated in FIG. 12 along with their full or partial compliments, or may for example use sequences known in the literature for cortisol or dopamine detection such as taught in Hariri, Amani A et al. “Modular Aptamer Switches for the Continuous Optical Detection of Small-Molecule Analytes in Complex Media.” Advanced materials (Deerfield Beach, Fla.) vol. 36,1 (2024): e2304410. doi: 10.1002/adma.202304410.

With further reference to FIGS. 8 and 9 and 13 where like numerals refer to like features, additional embodiments of the present invention are taught. Embodiments of the present invention may more generally include a first binding aptamer 827 that binds to the analyte and a second aptamer 825 that is at least partially complimentary to the first binding aptamer 827. In yet another embodiment of the present invention, a change in electron transfer between the redox tag 870, 970, 1370 and the electrode 820, 920, 1320 can be achieve by electrical conduction or lack of electrical conduction through the aptamers themselves. Strands of DNA consist of a phosphate-deoxyribose backbone connecting bases such as adenine and guanine and cytosine and thymine (or chemically modified or man-made bases). The bases are composed of aromatic rings that are spaced so closely in the double helix that the electron density of adjacent bases overlaps resulting in an electrically conductive pathway through the hybridized DNA. Two aptamers can come together similarly, for example as shown in FIG. 13 where when hybridized (no analyte) a redox tag such as methylene blue or other suitable redox tag 1370 will experience electron transfer through the hybridized aptamers to the electrode 1320. When the analyte binds then hybridization between the first and second aptamers 1325, 1327 is broken and electron transfer between the redox tag 1370 and electrode 1320 is reduced or eliminated. To maximize the signal to noise of such as sensor, generally a thicker blocking layer 1322 is desired such as C8, C10, C12, or even longer alkythiolate molecules or other suitable blocking layer materials. For example, a mercaptododecanol (C12) blocking layer may have 50× less electron transfer (redox current) than mercaptohexanol (C6) blocking layers. Electron transfer with the electrode 1320 is still required and can be enabled by having blocking layer defects supporing electron transfer that around or near the linker chemistry 1323, 1324, achieved for example by a mismatch in carbon length between linker chemistry 1323, 1324 and blocking layer molecules 1322, by different chemistries (such as charge, phenyle or other groups) or other suitable techniques. Alternately, linker 1323, 1324, or a single linker like that shown in FIG. 4 for element 424 can be made of an electrically conductive molecule such as a molecular wire such as conjugated molecules of [1,1′: 4′,1″-terphenyl]-4-methanethiol, [1,1′:4′,1″-terphenyl]-4-ethanethiol, or [1,1′:4′,1″-terphenyl]-4-propanethiol.

With further reference to embodiments of the present invention, a multi-bond aptamer can have a first aptamer that binds to the analyte, and a second aptamer which binds to at least one of the first aptamer and the analyte. Such first and second aptamers would bind to the analyte by layering where there is in part a sandwich of analyte bound to first aptamer, and first aptamer and optionally part of analyte bound to the second aptamer, referred to herein as a “multi-bond aptamer with layered aptamers.” To obtain such aptamers, aptamer selection (SELEX) can be performed for the first aptamer against the analyte, then SELEX repeated with the first aptamer and analyte but by introducing another library of second aptamers. The second aptamers are then down selected to those that bind to at least the analyte or first aptamer. Next the first aptamer is removed from SELEX and the second aptamers that no-longer bind to the analyte are then removed, leaving only second aptamer candidates that only bind when in the presence of both the analyte and the first aptamer. This is one further example of how to enable a device as taught herein where the second aptamer is signaling (and shorter and weaker in binding affinity) and the first aptamer is binding and non-signaling. Therefore, the present invention may include a device for detecting or measuring at least one large analyte in a sample fluid, the device comprising: at least one substrate; a sample fluid containing the analyte; and a plurality of aptamers capable of binding to the analyte, wherein the aptamers are physically bound to the substrate; wherein the aptamers include at least one tag capable of providing a signal and, wherein the aptamer has a change a change in geometry of the aptamer when the aptamer binds to the analyte, and the at least one tag has a resulting change in signal resulting from the analyte binding to the aptamer and associated change in geometry and wherein the aptamers further include at least a first portion and at least a second portion wherein when the first portion binds the analyte the second portion binds a least in part to the first portion.

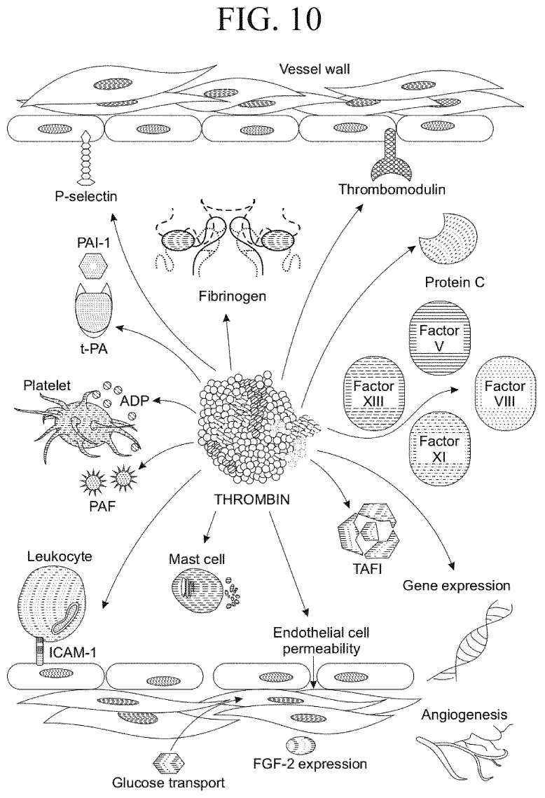

With reference to FIG. 10, an illustration of Thrombin is shown with the large variety of binding sites. This is instructive for the present invention, as aptamers have been developed for binding to many of these sites which can form FBPMA or SBPMAs of the present invention. The present invention may also have more than 2 binding sides, and a third binding portion of an aptamer (TBPA) is also possible, or even more, although not specifically taught herein. Additional binding sites may be required for particularly low concentration analytes that require very strong binding affinity (low kD values of nM, pM, or even fM).

With further reference to embodiments of the present invention, FIG. 11 is a list of additional FBPMA and SBPMA that may be useful in the present invention. Either Fragment 1 or Fragment 2 can be the FBPMA or SBPMA.

With further reference to embodiments of the present invention, FIG. 12 is a list of additional FBPMA and SBPMA that may be useful in the present invention. Either Fragment 1 or Fragment 2 can be the FBPMA or SBPMA

Examples of binding portions of aptamers can be derived from new aptamer selection or existing aptamer libraries for the large analyte insulin, such as those taught by White and colleagues in ACS Sens. 2019, 4, 498-503, ‘Electrochemical Aptamer-Based Sensor for Real-Time Monitoring of Insulin’. For example, the SLPMA portion and FBPMA portion may be Sequence 1:

SEQ. ID 1:
5′-HS-C6-AAAAGGTGGTGGGGGGGGTTGGTAGGGTGTCTTCTA.

A FLPMA can then include a plurality of thymine nucleotides.

The SBPMA can then be for example from Jennifer Y. Gerasimov, Cody S. Schaefer, Weiwei Yang, Rebecca L. Grout, Rebecca Y. Lai, ‘Development of an electrochemical insulin sensor based on the insulin-linked polymorphicregion’ Biosensors and Bioelectronics, Volume 42, 2013, Pages 62-68, ISSN 0956-5663

SEQ. ID 2:
ACAGGGGTGTGGGGACAGGGGTGTGGGG.

One of the first demonstrations ever for an electrochemical aptamer biosensor with a single binding site (e.g. FIG. 1), was for the large analyte molecule Thrombin as taught by Plaxco and colleagues in Angew. Chem. Int. Ed. 2005, 44, 5456-5459, ‘Label-free electronic detection of thrombin in blood serum by using an aptamer-based sensor’. Thrombin may also serve as an example application of the present invention by utilizing Sequence 3 and Sequence 4 for either a FBPMA portion or a SBPMA portion.

SEQ. ID 3:
GGTTGGTGTGGTTGG
SEQ. ID 4:
AGTCCGTGGTAGGGCAGGTTGGGGTGACT

For any large analyte (>1 kDa) the specific choice of FBPMA, SBPMA, optional SLPMA and FLPMA, are those that typically will provide two important performance parameters: (1) a Langmuir-isotherm binding response curve centered around the desired concentrations to be measured for large analyte; (2) a large or maximum change in sensor signal (sensor response) as large analyte binds to the multi-bond-aptamer, that is ideally at least one of >50%, >100%, or >200%. Although not specifically shown herein, a third binding site, binding portion of the aptamer, additional FLPMAs, or even more, may be utilized, referred to as a plurality of binding sites and binding portions of the aptamer.

Further examples for the FBPMA and SBPMA are shown in FIGS. 11 and 12 (and in the “Table of Fragments for additional FBMPA and SBMPA,” below), where either Fragment 1 or Fragment 2 can be the FBPMA or SBPMA. While these examples show optical tagging examples, optical tags can be modified for redox tags and the examples may also include linker chemistries to substrates such as gold (thiols), oxide (silanes) or other suitable linker/substrate combinations. The FBPMA and SBPMA can also be the same sequence, as for example using an aptamer that binds monomeric C-reactive protein to detect pentameric C-reactive protein which is made up of 5 linked monomeric units. Similarly, other analytes that display significant symmetry of two or more identical or nearly identical binding sites can use a FBPMA or SBPMA that are of the same sequence.

Many aptamers with good binding affinity to large analytes like proteins can likely be broken into fragments, and furthermore, near the end of SELEX rounds for aptamer selection SELEX could be stopped early such that dozens of candidate aptamers can be trialed. One way to ensure two fragments don't interact is to test their binding together, and separately, using one or more aptamer characterization techniques (surface plasmon, biolayer interferometry, isothermal calorimetry, circular dichroism, etc.).

TABLE of
Fragments for additional FBMPA and SBMPA

| | | SEQ. | | SEQ. |
|---|---|---|---|---|
| | | ID | | ID |
| Target | Fragment 1 | No. | Fragment 2 | No. |
| Thrombin | GGTTGGTG | — | TGGTTGG | — |
| | AGGTTGGGGTGACT | 5 | AGTCCGTGGTAGGGC | 6 |
| | GGTTGGTG | — | TGGTTGG | — |
| | GGTTGGTGTGGTTGG | 7 | AGTCCGTGGTAGGGG | 8 |
| | | | CA | |
| ATP | ACCTGGGGGAGTAT | 9 | TGCGGAGGAAGGT | 10 |
| | ACCTGGGGAGTAT | 11 | TGCGCAGGAAGGT | 12 |
| | ATTATAGCGGAGGAAA | 13 | AGTACCTGGGGAG | 14 |
| | GTATT | | TATATACT | |
| | ACCTGGGGGAGTAT | 15 | TGCGGAGGAAGGT | 16 |
| Adenosine | ACCTGGGGGAGTAT | 15 | ATGCGGAGGAAGGT | 17 |
| | AGAGAACCTGGGGGAG | 18 | TGCGGAGGAAGGTT | 19 |
| | TAT | | GAG | |
| | TACCTGGGGGAGTAT | 20 | TTGCGGAGGAAGGT | 21 |
| | CATCTCTTCTCCGAGCC | 22 | GAGCTGGAGGAAAA | 23 |
| | GGTCGAAATATTGGAGG | | AGTGACAA | |
| | AAGCTC | | | |
| Cocaine | TTCGTTCTTCAATGA | 24 | GGGAGTCAAGAACGAA | 25 |
| | AGTGGGACGACA | | | |
| | GGGAGTCAAGAAC | 26 | GTTCTTCAAT | 27 |
| | | | GAAGTGGGACGACA | |
| | GGGAGTCAAGAAC | 26 | GTTCTTCAATGAAGT | 28 |
| | | | GGGACGACA | |
| | GGGAGTCAAGAAC | 26 | GTTCTTCAATGAAGT | 28 |
| | | | GGGACGACA | |
| | GGGAGTCAAGAAC | 26 | GTCCTTCAACGAAGT | 29 |
| | | | GGGACGACA | |
| | CTCCTTCAACGAAGTG | 30 | GAGACAAGGAACAA | 31 |
| | GGTTCCTTCAACGAAGT | | GGAG | |
| | GGGTCTC | | | |
| | GTCCACTAACGAAGTTG | 32 | GGACGTGGAGACGT | 33 |
| | TCTTCCACTAACGAAGT | | GGAC | |
| | TGTCC | | | |
| Theo- | CGGCUGGGGCGAUACC | 34 | CCAGCCGAAAGGCCC | 35 |
| phylline | AGCCGAAA | | UUGGCACGUCGGG | |
| | AAAGGCGAUACCAGCC | 36 | GGCCCUUGGCAGCGUC | 37 |
| | GAAA | | AAAA | |
| Quinine | TCGTGGGACGACA | 38 | GGAGTCAAGAACGAA | 39 |
| | TTCGTTCTTCAATGA | | | |
| Quinine/ | GGGAGTCAAGAAC | 26 | GTTCTTCAATGAAGTG | 40 |
| Quinidine | | | GGACGACA | |
| ATP/ | CATCTCTTCTCCGAGCC | 41 | AAATAGTGGGTG | 42 |
| NAD⁺ | GGTCG | | | |

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A device for detecting or measuring at least one large analyte in a sample fluid, the device comprising:
at least one substrate; and
a plurality of multi-bond aptamers capable of binding to the analyte, wherein the aptamers are physically bound to the substrate;
wherein the multi-bond aptamers include at least one tag capable of providing a signal and, wherein the multi-bond aptamer has a change in geometry of the multi-bond aptamer when the multi-bond aptamer binds to the analyte, and further, wherein the at least one tag has a resulting change in signal resulting from the analyte binding to the multi-bond aptamer and associated change in geometry.

2. The device of claim 1, wherein the multi-bond aptamer has two or more binding portions.

3. The device of claim 1, wherein the tag is a redox tag.

4. The device of claim 3, wherein the substrate is an electrode.

5. The device of claim 1, wherein the tag is an optical tag.

6. The device of claim 5, wherein the substrate is an optical waveguide.

7. The device of claim 1, wherein the plurality of multi-bond aptamers further comprise at least one first binding portion and at least one second binding portion.

8. The device of claim 7, wherein one or more of the plurality of multi-bond aptamers further comprise at least one flexible linking portion.

9. The device of claim 7 wherein the first binding portion and the second binding portion are separated by at least one connection of molecules, and further, wherein the at least one connection of molecules comprises at least one of:
a. molecules not binding to the analyte during binding of the multi-bond aptamer to the target analyte;
b. at least one molecule that attaches at least the first binding portion and at least the second binding portion to the substrate;
c. a separation resulting from the first portion and the second portion having independent binding chemistry linkages to the substrate on which they are bound.

10. The device of claim 7 wherein the first binding portion and the second binding portion are bound to the substrate and are further bound to each other.

11. The device of claim 10 wherein the first binding portion and the second binding portion are bound to the substrate and are further bound to each other by base-pair matching between a plurality of base pairs.

12. The device of claim 7 wherein at least one of the first binding portion or the second binding portion form a stem loop in absence of analyte binding to the multi-bond aptamer.

13. The device of claim 1 wherein the device has a sensor response, and the sensor response is a percentage selected from the group consisting of >50%, >100%, and >200%.

14. A device for detecting or measuring at least one large analyte in a sample fluid, the device comprising:
at least one substrate; and
a plurality of aptamers capable of binding to the analyte, wherein the aptamers are physically bound to the substrate;
wherein the aptamers include at least one tag capable of providing a signal and further, wherein the aptamer has a change in geometry of the aptamer when the aptamer binds to the analyte, and the at least one tag has a resulting change in signal resulting from the analyte binding to the aptamer and associated change in geometry;
and further, wherein the aptamers further include at least a first portion with at least one tag that does not bind to the analyte, and at least a second portion which does bind to the analyte.

15. The device of claim 14 wherein the first portion has a weaker binding affinity than the second portion and the first portion is shorter in length than the second portion.

16. The device of claim 14 wherein the first portion has at least one tag and forms a stem loop in absence of binding of analyte to the second portion.

17. The device of claim 14, wherein the tag is a redox tag.

18. The device of claim 17, wherein the substrate is an electrode.

19. The device of claim 14, wherein the tag is an optical tag.

20. The device of claim 19, wherein the substrate is an optical waveguide.

21. The device of claim 14, further comprising at least one flexible linking portion between the first portion and the second portion.

22. The device of claim 14 wherein the first portion and the second portion are separated by at least one connection of molecules, wherein the at least one connection of molecules comprises at least one of:
a. molecules not binding to the analyte during binding target analyte;
b. at least one molecule that attaches at least the first portion and at least the second portion to the substrate;
c. a separation resulting from the first portion and the second portion having independent binding chemistry linkages to the substrate on which they are bound.

23. The device of claim 22 wherein at least the first portion and at least the second portion are bound to the substrate and are further bound to each other.

24. The device of claim 22 wherein at least the first portion and at least the second portion are bound to the substrate and are further bound to each other by base-pair matching between a plurality of base pairs.

25. The device of claim 14 wherein the device has a sensor response, and the sensor response is a percentage selected from the group consisting of >50%, >100%, or >200%.

26. The device of claim 1 wherein the multi-bond aptamer is further comprised of at least a first portion and a least a second portion, and further, wherein when the first portion binds the analyte the second portion binds a least in part to the first portion.

27. The device of claim 14 wherein when the first portion binds the analyte the second portion binds a least in part to the first portion.

28. A method for detecting or measuring at least one large analyte in a sample fluid, the method comprising exposing the sample fluid to the device of claim 1 and detecting or measuring the amount of the large analyte using data regarding changes in one or more aptamer parameters.

29. The method of claim 28 wherein the sample fluid is exposed to the device by placing the device in direct contact with the sample fluid or placing the device in fluid communication with the sample fluid.

30. A device for detecting or measuring at least one analyte in a sample fluid, the device comprising:
at least one substrate; and
a plurality of aptamer pairs, each aptamer pair comprising a first aptamer and a second aptamer, wherein at least the first aptamer or second aptamer of each of the plurality of aptamer pairs are bound to the substrate;
wherein the first aptamer includes at least one tag, and wherein the second aptamer is capable of binding the at least one analyte
wherein, in each aptamer pair, the first aptamer and second aptamer are sufficiently complementary to hybridize to one another in the absence of analyte binding to the second aptamer, and wherein signal from the tag to the substrate occurs through the hybridized first and second aptamers; and
wherein, when analyte binds to the second aptamer, the first aptamer and second aptamer separate to a degree that reduces or eliminates signal from the tag to the substrate.

31. The device of claim 30, wherein the second aptamer includes at least a first binding portion and a second binding portion, wherein the first and second binding portions are capable of binding to the at least one analyte.

32. The device of claim 30, wherein the second aptamer includes more than two binding portions, wherein each of the binding portions are capable of binding to the at least one analyte.

33. The device of claim 30, wherein the substrate includes an electrode.

34. The device of claim 33, wherein the tag is a redox tag.

35. The device of claim 30, wherein the first aptamer and/or second aptamer is directly bound to the substrate.

36. The device of claim 30, wherein the first aptamer and/or second aptamer is indirectly bound to the substrate.

37. The device of claim 30, wherein the device is capable of a sensor response, and the sensor response is a percentage selected form the group consisting of >50%, >100%, and >200%.

38. A method for detecting or measuring at least one analyte in a sample fluid, the method comprising exposing a sample fluid to the device of claim 30 and detecting or measuring the amount of the analyte using data regarding changes in one or more parameters of the first and/or second aptamers.

39. The method of claim 38, wherein the data regarding changes in one or more parameters of the first and/or second aptamers is provided by a change in signal between the tag and the substrate.

40. The method of claim 38, wherein the sample fluid is exposed to the device by placing the device in direct contact with the sample fluid or placing the device in fluid communication with the sample fluid.
